(19) United States
(12) Patent Application Publication
(10) Pub. No.: US 2005/0276743 A1
(43) Pub. Date: Dec. 15, 2005

(54) Method for fabrication of porous metal templates and growth of carbon nanotubes and utilization thereof

(76) Inventors: Jeff Lacombe, Reno, NV (US); Glenn Sklar, Reno, NV (US); Manoranjan Misra, Reno, NV (US); Krishnan S. Raja, Reno, NV (US); Shantanu Namjoshi, Reno, NV (US)

Correspondence Address: NIXON PEABODY, LLP, 401 9TH STREET, NW, SUITE 900, WASHINGTON, DC, 20004-2128, US

(21) Appl. No.: 11/033,839

(22) Filed: Jan. 13, 2005

Related U.S. Application Data
(60) Provisional application No. 60/535,804, filed on Jan. 13, 2004.

(51) Int. Cl. A01H 1/00; C25D 5/34

(52) U.S. Cl. 423/447.3; 205/219; 205/324

(57) ABSTRACT

The present invention relates to controlled growth of carbon nanotube (CNT) arrays via chemical vapor deposition (CVD) using novel porous anodic aluminum oxide (AAO) templates, which have been seeded with transition metal catalysts. The resulting CNT bundles may be dense and long and can be used for numerous applications. Further, the porous AAO templates and the CNTs grown thereby, can be functionalized and used for separation of chemical species, hydrogen storage, fuel cell electrocatalyst and gas flow membranes, other catalytic applications, and as a bulk structural material.

This application claims the benefit of U.S. Provisional Patent Application No. 60/535,804, filed on Jan. 13, 2004, which is hereby incorporated by reference for all purposes as if fully set forth herein.

### BACKGROUND OF THE INVENTION

1. Field of the Invention

The present invention relates to controlled growth of carbon nanotube (CNT) arrays via chemical vapor deposition (CVD) using novel porous anodic aluminum oxide (AAO) templates, which have been seeded with transition metal catalysts. The resulting CNT bundles may be dense and long and can be used for numerous applications. Further, the porous AAO templates and the CNTs grown thereby, can be functionalized and used for separation of chemical species, hydrogen storage, fuel cell electrocatalyst and gas flow membranes, other catalytic applications, and as a bulk structural material.

2. Discussion of the Related Art

CNTs have been proposed as new materials for scanning probe microscope tips, gas and electrochemical energy storage, one-dimensional quantum wires with either semiconductor or metallic behaviors (depending on geometrical parameters), hydrogen storage, chemical sensors, microelectronic devices, catalyst supports, and molecular-filtration membranes. Also, work has been done on electron emission from carbon nanotubes for use in cold cathode, flat panel displays, because of their good emission stability, long emitter lifetime, and the potential to operate at low voltages. There are numerous advantages of using CNTs as a field emission material these advantages include a high aspect ratio, high chemical inertness, good mechanical strength, and tips with small radius of curvature.

Most of these applications require a fabrication method capable of producing uniform CNTs with well-defined and controllable properties. The ability to control the dimension, location, and structure of CNTs will allow the reliable study of their physical properties and use in the examples above.

In one related art approach, CNTs are grown directly from catalysts without the use of a template. Unfortunately, in this technique the CNTs have a high degree of contamination and the CNTs are not well aligned. More specifically, in this technique the CNT arrays are held together by Van Der Waals forces. This gives vertical alignment and confinement of CNTs defined by the patterned catalyst boundaries. Unfortunately, the CNTs produced by these methods are weakly bound to the substrate, therefore, further processing and integration into devices is not possible.

In comparison, in other related art approaches CNTs are grown inside AAO templates to allow for movement and integration of the aligned CNT arrays into devices without damage as these CNT arrays are held together by the AAO template structure.

FIG. 1 illustrates a perspective view of a related art of a carbon nanotube array grown from the porous aluminum oxide template of the related. Referring to FIG. 1, illustrating an AAO template 100, CNTs 102 are grown perpendicular to a rigid substrate 104 (e.g., Al₂O₃). More specifically, the process for making the substrate requires forming the oxide template, aluminum anodizing to form pores 103, and conventionally depositing a catalyst 106 (e.g., AC electrodeposition). The electrodeposited metal oxide is reduced to the catalyst metal. CNTs 102 are then formed by pyrolysis of acetylene or ethylene.

Table 1 illustrates the related art of CNTs grown via AAO templates, for example, CNTs growing beyond the AAO pore confines (as opposed to CNTs stopping at the AAO pore mouths). Table 1 reports CNTs relatively low growth rates, and CNT lengths of 1-90 microns beyond the AAO pore mouths from CVD times of 20-120 minutes with hydrocarbon feedstock. Additionally, Table 1 reports CNTs growth rates of 120-720 minutes with carbon monoxide feedstock. Using CO a catalyst as a growth gas produced more highly graphitized CNTs at a slower rate.

TABLE 1
Related Art, Summary of AAO templated carbon nanotube growth reported in the literature.

| Template | | | | | Catalyst Reduction | | | | Chemical Vapor Deposition | | | |
|---|---|---|---|---|---|---|---|---|---|---|---|---|
| | | depth to | catalyst | unfilled | | flow | | | | flow | | |
| | | catalayst | diameter | diameter | reducing | rate | time | temp | growth | rate | time | temp |
| Ref | catalyst | μm | nm | nm | gas | sccm | Hr | C | gas | sccm | min | C |
| 1 | Co | 0.05 | 55 | 75 | NA | 100 | NA | NA | CO | 100 | 720 | 700 |
| 2 | Co | 0.16 | 29 | 65 | NA | 100 | NA | NA | CO | 100 | 720 | 600 |
| 3 | Co | 0.8 | 50 | 50 | H₂ | 10 | 1 | 500 | CO | 50 | 120 | 650 |
| 3 | Co | 7.6 | 74 | 74 | H₂ | 10 | 1 | 500 | H₂/C₂H₂ | 40/20 | 20 | 650 |
| 4 | Co | 0.9 | 80 | 80 | H₂ | 10 | 1 | 500 | H₂/C₂H₂ | 40/20 | 20 | 650 |
| 5 | Co | 0.9 | 80 | 80 | H₂ | 10 | 1 | 500 | H₂/C₂H₂ | 40/20 | 40 | 650 |
| 6 | Co | 0.9 | 75 | 75 | H₂ | 2 | 1 | 600 | H₂/C₂H₂ | 40/20 | 15 | 650 |
| 7 | Co | 0.4 | 50 | 50 | H₂ | 2 | 1 | 750 | H₂/C₂H₄ | 2/2 | 60 | 750 |
| 8 | Co | 0.6 | 29 | 88 | CO | 100 | 4 | 600 | C₂H₂ | 10 | 20 | 700 |
| 3 | Co | 0.8 | 74 | 74 | H₂ | 10 | 1 | 500 | C₂H₂ | 4 | 120 | 650 |
| 9 | Co | 4.9 | 40 | 40 | CO | 100 | 4 | 600 | C₂H₂ | 10 | xx | 700 |

| | SEM Characterization | | | | | TEM Characterization | | | | | |
|---|---|---|---|---|---|---|---|---|---|---|---|
| | | beyond | length | diameter | | | | | wall | number | interwall |
| | CNTs stop at | mouth | out | out | CNT | catalyst | Graph- | CNT | thick- | of | spacing |
| Ref | Pore mouth | % | μm | nm | tips | fate | itization | core | ness | shells | nm |
| 1 | no | 100 | 11 | 60 | closed | xx | G | H | 18-20 | 50-55 | xx |
| 2 | no | 100 | 7 | 32 | closed | xx | G | H | xx | xx | xx |
| 3 | no | 10 | 1 | 50 | closed | M-T | G | H | 18-20 | 50-55 | xx |
| 3 | no | 20 | 1-60 | 75 | closed | M-T | G | H | 30-35 | 80-90 | xx |
| 4 | no | 25 | 1-90 | 80 | xx | xx | G | H | 14-17 | 40-50 | 0.35 |
| 5 | no | 25 | 1-90 | 80 | xx | xx | G | H | 16-20 | 45-55 | 0.35 |
| 6 | no | xx | 1-6 | 75 | xx | | | | | | |
| 7 | no | xx | 1-90 | <45 | xx | | | | | | |
| 8 | no | xx | 1-10 | 60 | xx | | | | | | |
| 3 | no | 10 | 1-2 | <75 | xx | | | | | | |
| 9 | no | xx | xx | <40 | xx | | | | | | |

G = graphtic, H = hollow, M = middle, T = top
1 J.S. Lee, G.H. Gu, H. Kim, J.S. Suh, I. Han, N.S. Lee, J.M. Kim, and G.S. Park, Synthetic Metals 124 (2001) 307-310, which is hereby incorporated by reference as if fully set forth herein.
2 Jin Seung Lee and Jung Sang Suh, Journal of Appiled Physics 92, #12 (2002) 7519-7522, which is hereby incorporated by reference as if fully set forth herein.
3 Hee-Young Hwang, Thesis from Pohang University of Science and Technology, Pohang, Korea (2000), which is hereby incorporated by reference as if fully set forth herein.
4 Soo-Hwan Jeong, Ok-Joo Lee, Kun-Hong Lee, Sang-Ho Oh, and Chan-Gyung Park, Chemistry of Materials 14, #10 (2002) 4003-4005, which is hereby incorporated by reference as if fully set forth herein.
5 Soo-Hwan Jeong, Ok-Joo Lee, Kun-Hong Lee, Sang-Ho Oh, and Chan-Gyung Park, Chemistry of Materials 14, #4 (2002) 1859-1862, which is hereby incorporated by reference as if fully set forth herein.
6 Soo-Hwan Jeong, Hee-Young Hwang, Kun-Hong Lee, Yongsoo Jeong, Appiled Physics Letters 78, #14 (2001) 2052-2054, which is hereby incorporated by reference as if fully set forth herein.
7 Tatsuya Iwasaki, Talko Motol, and Tohru Den, Applied Physics Letters, 75, #14 (1999) 2044-2046, which is hereby incorporated by reference as if fully set forth herein.
8 Jin Seung Lee, Geun Hoi Gu, Hoseong Kim, Kwong Seok Jeong, Jiwon Bae, and Jung Sang Suh, Chemistry of Materials 13, #7 (2002) 2387-2391, which is hereby incorporated by reference as if fully set forth herein.
9 D.N. Davydoy, P.A. Sattari, D. AlMawlawi, A. Oslka, T.L Haslett, and H. Moskovits, Journal of Applied Physics 86, #7 (1999) 3983-3987, which is hereby incorporated by reference as if fully set forth herein.

In the related art processes, as shown from Table 1 and references 1-9 above, which are hereby incorporated by reference as if fully set forth herein, the catalyst was formed in the AAO pores using AC electrodeposition from a cobalt sulphate/boric acid/ascorbic acid solution, 60-100 Hz. More specifically, in references [1-2] (hereinafter “Lee, et al.”) a follow-up process using two additional subsequent chemical steps to etch back cobalt from overfilled AAO pores and then widen the pore section above the catalyst 108 as shown in FIG. 1 where conducted. By doing so, Lee, et al., [1, 2], reported nearly 100% pore emergence of CNTs. Unfortunately, this technique requires a large number of processing steps and is complex to implement on a large scale.

As shown from the foregoing and Table 1, there is a need to grow CNTs that have a high degree of purity (e.g., well-graphitized), high growth rate, CNTs that are long and continuous, and economically formed with a simplified process.

### SUMMARY OF THE INVENTION

Accordingly, the invention is directed to a method for fabrication of porous metal templates and growth of carbon nanotubes and utilization that substantially obviates one or more of the problems due to limitations and disadvantages of the related art.

An advantage of the invention is to provide a carbon nanotubes structures having large diameter.

Another advantage of the invention is to provide a simplified method for producing carbon nanotubes and carbon nanotube structures.

Yet another advantage of the invention is to provide a controlled method for forming well-graphitized carbon nanotubes.

To achieve these and other advantages and in accordance with the purpose of the invention, as embodied and broadly described, an anodized aluminum oxide template for carbon nanotube growth including an anodized aluminum substrate having a plurality of pores arranged in the anodized aluminum substrate. A plurality of catalyst particles are arranged substantially uniformly in the plurality of pores to provide nucleation sites for carbon nanotube growth. A plurality of carbon nanotubes are arranged within the pores and a metal material is arranged over the anodized aluminum substrate and covering a plurality of carbon nanotubes.

In another aspect of the invention, a method of fabricating anodized aluminum oxide template includes annealing an aluminum material. The annealed aluminum material is electropolished. A first anodizing of the aluminum material is performed and the first anodized material is removed. A second anodizing of at least a portion of the aluminum material is performed to form an anodized aluminum layer and to form a plurality of pores in the anodized aluminum layer. A plurality of catalyst particles are arranged substantially uniformly in the plurality of pores to provide nucleation sites for carbon nanotube growth via electrodeposition. Carbon nanotubes within the plurality of pores are formed and metal layer is formed on the anodized aluminum layer covering the carbon nanotubes. In this aspect of the invention, a kit may also be formed to enable another use to subsequently grown carbon nanotubes.

In yet another aspect of the invention, forming carbon nanotubes includes providing an aluminum material having a first surface and a second surface opposite said first surface. Anodizing the first surface of the aluminum material to form a plurality of pores and depositing a catalytic material into the plurality of pores to provide nucleation site for a first set of carbon nanotubes and a second set of carbon nanotubes at bottom of the plurality of pores. The first set of carbon nanotubes are grown from the catalytic metal to reach about the top portion of the plurality of pores. A metal layer is formed over the anodized aluminum material and covering the first set of carbon nanotubes, followed by forming a protective barrier layer over the metal layer. A portion of the aluminum material is removed on the second surface to expose at least a bottom portion the catalyst metal. A second set of carbon nanotubes are grown at the exposed portion of the catalytic metal.

In still another aspect of the invention, a method of fabricating anodized aluminum oxide template, forming an aluminum material on the substrate. Anodizing at least a portion of the aluminum material to form an anodized aluminum layer and to form a plurality of pores in the anodized aluminum layer. Pulse-reversed electrodepositing a plurality of catalyst particles into a plurality of pores to provide nucleation sites for carbon nanotube growth.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention.

In the drawings:

FIG. 1 illustrates a perspective view of a related art of a carbon nanotube array grown from the porous aluminum oxide template;

FIG. 2 illustrates is a FE-SEM micrograph of Anodic Aluminum Oxide (AAO) template with pores according to an embodiment of the invention;

FIG. 3 illustrates a process steps A-H according to another embodiment of the invention;

FIG. 4 illustrates a cross-sectional view of AAO template with internal pores for assembling CNTs according to another embodiment of the invention;

FIG. 5 illustrates a Self-Assembled Array of CNTs with carbon spacers/connectors, after removal of the AAO template according to another embodiment of the invention;

FIG. 6 illustrates a cross-sectional view of a template having pore density gradient for production of Y-type CNTs according to another embodiment of the invention;

FIG. 7 illustrates macroscopic CNT growth including carbon nanostructures according to another embodiment of the invention;

FIG. 8(a) illustrates a SEM image of templated CNTs at 300× according to FIG. 7;

FIG. 8(b) illustrates a SEM image of templated CNTs at 100,000× according to FIG. 7;

FIG. 8(c) illustrates a SEM image of templated CNTs at 699,000× according to FIG. 7;

FIG. 8(d) illustrates a SEM image of FIG. 8(c) at 100,000× showing an end portion of a carbon nanostructure;

FIGS. 9(a)-9(d) illustrate a multilayered template according to another embodiment of the invention; and

FIGS. 10(a)-10(b) illustrate an apparatus and method to produce continuous carbon nanotubes according to another embodiment of the invention.

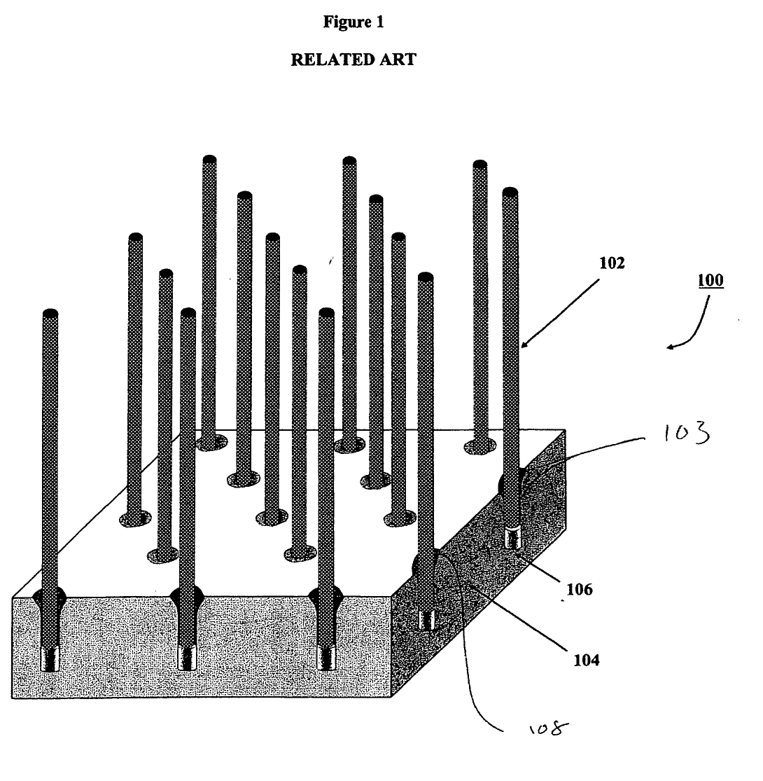

### DETAILED DESCRIPTION OF THE ILLUSTRATED EMBODIMENTS

The invention relates to a method and apparatus for growing of carbon nanotubes (CNTs) on transition metal catalysts by chemical vapor deposition (CVD) conducted via refined anodic aluminum oxide (AAO) templates. Optionally, the templates may be formed from aluminum layers on substrates, such as, for example, plastic, semiconductor, metal, and combinations thereof. The invention produces dense, solid, continuous, and long CNTs. These CNTs may be used for number applications including hydrogen storage, quantum wires, molecular filtration, catalytic applications, and as a monolithic bulk structural material.

More specifically, a smooth carbon nanotube structure prepared according to the invention showed a diameter that was larger than the pores of the AAO template. For example, it exhibited a rope like shape. It is thought that these structures are a convergence of a plurality of CNTs grown out of the pores and/or from the surface of the AAO template.

It is generally known in the art that the growth of CNTs via AAO templates using the CVD process depends upon parameters, such as: (a) the pore diameter, pore length, and interpore distance of the AAO template; (b) metal catalyst electrodeposition method and conditions; (c) uniformity of catalyst deposit; (d) depth from template surface to catalyst; (e) catalyst reduction conditions; (f) chemical etching of catalyst and/or AAO pore walls; (g) CVD gas or gas mixtures, gas flow rates, gas pressures, CVD temperature profile, and reaction time. The characteristics of the resulting CNTs and the applications possible may be extended by chemical functionalization of the resulting CNTs. Various applications are also possible by chemical functionalization of the AAO templates themselves (e.g., with or without CNTs present).

More specifically, functionalization includes doping of the CNTs and/or AAO templates with substances, including numerous chemicals, dopants, the like, such as known in the art. For example, the doping of the AAO pores walls may be conducted before or after the growth of CNTs. For example and illustrative purposes, in hydrogen storing applications, the CNTs may be doped with Mg, Na, Li; for metallic functionalization dopants may include Au, Pt, Ag, Fe, Ni, other transition metals, and the like; and for semiconductors applications dopants may include N-type and P-type dopants. Of course other types of functionalization are possible as known in the art.

Anodized Aluminum Oxide Template with Cobalt Catalyst

The structure, length, and diameter of CNTs grown via AAO templates by CVD are controlled by the AAO template microstructure, the deposition of transition metal catalysts, and the CVD conditions employed.

In one aspect of the invention, AAO templates of the invention were prepared using a controlled method to fabricate templates involving: 1) electropolished aluminum; 2) two-step anodization process; 3) pulse-reversed electrodeposition of cobalt or other transition metals metal catalysts. The catalysts include, metals, such as, transition metals, Fe, Ni, Co. The catalysts also include organometallics, like nickelocene, ferrocene, and the like, which are decomposed to the metal prior to CNT growth. The two step anodization process is described in detail in Nielsch, et. al., Uniform Nickel Deposition into Order Alumina Pores by Pulsed Electrodeposition, Adv. Mater., 2000, 12. No. 8, which is hereby incorporated by reference as if fully set forth herein. More specifically, an example of a detailed procedure to produce nanostructured AAO template having an area of about 1 cm² and being about 1 micron long pores for CNT growth is given below.
1. Anneal aluminum under helium or argon at about 500° C. for about 3 hours.
2. Electropolish in perchoric acid/ethanol at a ratio of about 1:3, at about 20 V DC, for about 2-5 min., at about 7° C., other ratios are also possible, for example, 1:4.
3. Anodize in about 0.3 M oxalic acid, at about 40 V DC, for about 4-12 hours, at about 17° C.
4. Remove anodized coating of step 3, for example, removing the coating in chromophosphoric acid at about 40-60° C., for about 1-2 hours.
5. Anodize in about 0.3 M oxalic acid, at about 40 V DC, for about 8 min., at about 17° C.
6. Widening of AAO pores to about 50 nm in diameter by about 0.3M oxalic acid, at about 30° C., for about 3 hours.
7. Barrier layer thinning can be done by current control. For example, barrier layer thinning in about 0.3 M oxalic acid, with about a 12 minute declining current controlled ramp starting at about 1.2 mA/cm² and finishing treatment at about 0.1 mA/cm² at about 17° C., in order to thin the dense oxide, electrically insulating, barrier layer to about 5-10 nm to improve the electrical conductivity for the pulsed electrodeposition step.
8. Computer controlled pulse-reverse electrodeposition, and optionally employing simultaneous current and voltage control, of cobalt from pore bottoms on up.
9. Templates ready for CNT growth.

In this process, the AAO pore diameter is determined by steps 3, 5, and 6, is chosen depending on the CNT desired diameter. More specifically, the larger pore size gives larger CNT diameters. Thus, this process allows for the control of pore size. Also, smooth CNT structures are grown from the pores and/or surface of the template having a large diameters.

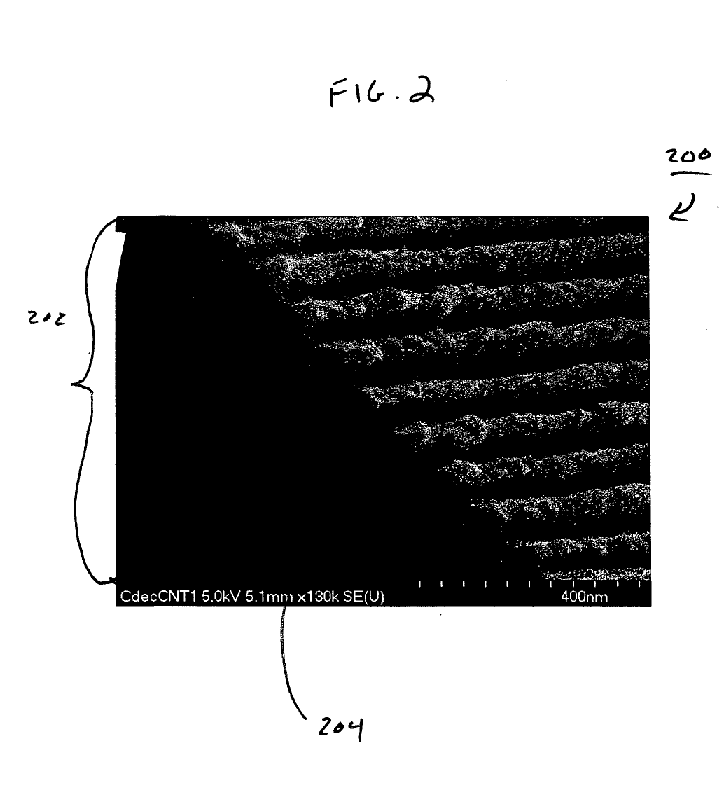

FIG. 2 illustrates a SEM image of an AAO template which is ready for CNT growth. The average pore diameter of these AAO templates was controlled to be in the range from about 25-50 nm, with an average interpore center-to-center distance of about 100 nm, and a pore density of about 10¹⁰/cm². The nanopores are uniformly and homogenously formed throughout the AAO template. In one aspect of the invention, the pore diameters are about 50 nm. Section 202 illustrates nanopores without catalyst particles. Metallic cobalt catalyst particles were then deposited via computer controlled pulse-reverse electrodeposition into the pores 204, which was described above. This technique may be performed with tools known in the art.

More specifically, in the computer controlled pulse-reverse electrodeposition, for example, using simultaneous control of the current and voltage, were performed to allow for a cathodic waveform and pulse time designed to deposit catalyst starting at the AAO pore bottoms. The cathodic pulse is timed to stop before the metal ions in solution at the existing plated metal/metal solution interface are exhausted, preventing hydrogen formation, which would have an ill effect on the catalyst deposit. An anodic waveform and pulse time was designed to selectively de-plate any impurities from the just plated layer of metal to leave only metal particles of a desired grain size range, and keep the metal face clean for the next cathodic pulse to lay down the next layer with the same crystallographic orientation. The anodic pulse also discharges the capacitance of the barrier layer to immediately stop metal deposition. Finally, a rest time of up to about one second is allowed before the next cathodic pulse to replenish metal ions at the plated metal/metal solution interface. This type of plating was used in Nielsch, et. al., Uniform Nickel Deposition into Order Alumina Pores by Pulsed Electrodeposition, Adv. Mater., 2000, 12. No. 8, which is hereby incorporated by reference as if fully set forth herein, for the complete filling of AAO pores with single crystal nanowires of nickel, cobalt, or iron for research on photonic crystals and magnetic studies. The invention utilizes pulse reverse-electrodeposition for CNT growth, which does not appear in the literature for CNT growth. More specifically, in the related art, AC electroplating is used as shown in Table 1, which requires additional processing steps.

Modified Anodized Aluminum Oxide Template and CNT Growth System

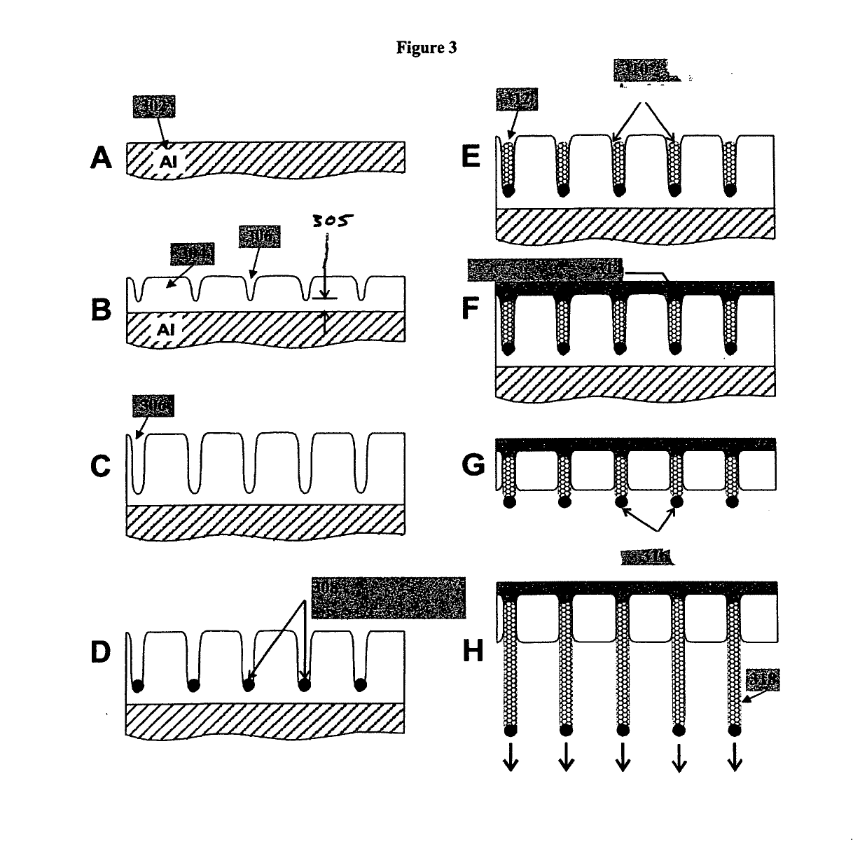

FIG. 3 illustrates a process steps A-H according to another embodiment of the invention. This method produces a novel AAO template and provides a CNT growth system which permits CNT growth at substantially the same rate from all of the AAO pores. This method provides a means for synthesis of aligned arrays of long, continuous, CNTs of equal length. This capability is achieved by carefully controlling the catalysis process so that CNT growth occurs at the tip, allowing for this synthesis, rather than at the base of the CNTs (i.e., at the top of the “forest” of CNTs, rather than at the roots), thereby, avoiding the transport limitations associated with gas permeation through the CNT forest to the bases of the CNTs at a rate sufficient to sustain a constant CNT growth rate. This positions (or fixes) catalyst particles at the CNT tips. With the catalytic particles located at the tips of the tubes, the gas permeation process is not expected to be a constraining factor as the CNTs increase in length.

As described earlier, the process begins with the preparation of an anodic aluminum oxide (AAO) template, with regular and controllable pore sizes and spacings, and with carefully placed catalytic materials. With this initial step completed, additional steps place the catalyst in a position where it will continually be exposed to the carbon feedstock gas.

Referring to FIG. 3, in step A, a commercially available high purity aluminum material 302 (e.g., preferably 99.999%) is utilized as the substrate. The substrate in this embodiment is aluminum. Optionally, the aluminum material may be formed on number of different substrates, such as, for example, plastic, semiconductor, metal, and combinations thereof.

In step B, the AAO template 304 is formed on the aluminum material 302 as described previously herein. The barrier layer thickness is represented as 305. More specifically, anodization of aluminum to form AAO pores 306 as described herein throughout.

In step C, chemical pore widening is completed. For example, widening of AAO pores 306 to about 50 nm in diameter by about 0.3M oxalic acid, at about 30° C., for about 3 hours.

In step D, catalytic metals 308 (such as, for example, cobalt) are deposited into pores 306. More specifically, the catalyst metal 308 may be electrochemically deposited in the pores via computer controlled pulse-reverse electrochemical deposition. Some of the catalytic material was formed on top of the AAO template, thereby enabling fast growth rate of carbon nanostructures.

In step E, CNTs 310 are grown in pores 306 via CVD, for example, the CNTs 310 are grown with CVD using carbon monoxide at about 100 sccm and at a temperature of about 650° C. for about 1 minute or longer. This enables growth of well-graphitized CNTs (e.g., carbon atoms arranged in perfect hexagonal network giving CNT material with desired electrical and mechanical properties) at the pore bottoms with a length of about 100 nm or more depending on deposition conditions. More specifically, with a longer deposition the well-graphitized CNTs are formed to a predetermined distance either just emerging form the AAO pores to just under the AAO pores. This will allow for electrical conductivity between the CNTs and/or individual CNTs with the subsequently formed metal layer described in step F, below. Optionally, the CNT growth gas is changed to about 20 sccm of acetylene and about 180 sccm of argon, to fill the rest of the AAO pore 306, thereby growing poorly graphitized (e.g., disordered carbon network) carbon nanostructure material (not shown) which stops at the AAO pore mouths 312, by virtue of being catalyzed by the AAO pore walls. In this case, the poorly-graphitized material (not shown) may serve as an anchor for subsequently formed CNTs in step H.

In step F, the top of the pore structure is coated with an electrically conductive material 314 that protects the AAO template 304 from subsequent processing steps and provides a contact to allow the final CNTs to be individually addressed if integrated into an electrical device. That is, the electrically conductive material 314 provides an ohmic contact for the CNTS 310. More specifically, the electrically conductive material 314 is applied over the surface, covering the CNTs 310 and the AAO template 304. The conductive material 314 may be any type of noble metal, transition metals, semiconductor, and the like, formed by known techniques in the art. In one aspect of the invention, the electrically conductive material 314 was formed of metal to about 10 nm with sputtering techniques known in the art, for example, nickel, platinum, or chromium, and/or combinations thereof, were sputtered on the surface of the AAO template 304 and in electrical contact with the well-graphitized CNTs 310. Optionally, this layer may be built up by ordinary nickel electroplating to a thickness of about 100 μm to further sustain the integrity of the AAO template 304 throughout the remaining process steps.

However, when the optional step of changing growth gases in step E is performed and the poorly-graphitized material is formed adjacent to the subsequently deposited electrically conductive layer 314, the poorly-graphitized material may not serve as an adequate conductor to reach the subsequently formed well-graphitized CNTs 318, of step H. Nevertheless, the electrically conductive layer 314 may be formed to provide at least a structural support for the overall structure.

In step G, the remaining Al material 302 and the remaining barrier layer 305 of the AAO 304 template is removed on the reverse side, leaving behind the catalyst particles 316 arranged on the ends of the partially exposed the well-graphitized CNTs. More specifically, the remaining barrier layer 305 and the remaining Al material 302 are selectively removed with techniques known in the art, for example, plasma etching, wet etching, and the like. Additionally, the electrically conductive material 314 is covered, for example, with a protective coating, for example, nail polish (not shown) to protect the electrically conductive material 314. Of course, other types of protective coatings are possible, for example, insulating organic materials, such as, oxides, epoxy resins, lacquers, insulating polymers, and the like, formed by known techniques in the art. Moreover, additional and other protective coatings are also possible.

In a subsequent process, the protective layer is removed and devices are formed on or integrated with the electrically conductive layer 314. For example, these devices may include switching devices, memory devices, display devices, and the like. The switching devices, may include for example, thin film transistors, field effect transistors, and the like, as known in the art. Moreover, the display devices may include liquid crystal displays, plasma displays, organic light emitting diode displays, and the like. Moreover, other types of devices may also be formed on the conductive layer or integrated with the electrically conductive layer 314 and in electrical connection with the subsequently formed CNTs 318, in step H.

In this embodiment, and by way of example, the reverse side of the template 300, which is aluminum metal 302 and generally comprises about >99% of the original template thickness, is selectively removed, for example, removed chemically in acidic copper chloride solution. The remaining barrier layer 305 of about 10 nm and about 100 nm of the AAO original pore bottoms is selectively removed in phosphoric acid. Thereby, leaving exposed the catalyst particles at the tips of the CNTs 316. The nail polish (not shown) protecting the electrically conductive layer 314 is removed in acetone.

In step H, a CVD process is applied to these newly exposed, previously grown, CNT tips, pushing the catalyst particles 316 along as the CNTs 318 grow longer. This maintains the supply of carbon feed stock gas access to the catalyzed regions, and results in long, continuous growth, emanating from 100 percent of the AAO template pores. More specifically and by way of example, the CVD growth process is resumed on these newly exposed CNTs tips 316 where the active growth sites are now at the CNT tips 316 (i.e., where the catalyst particles are located).

In this embodiment and by way of example, the CVD conditions are about 650° C. with about 100 sccm of carbon monoxide for well-graphitized CNTs at a growth rate of about 10 nm per minute. Optionally, about 20 sccm of acetylene with about 40 sccm of hydrogen for a faster growth rate of fairly well-graphitized CNTs at about 1 μm per minute. This CNT growth step can be continued for long growth times with the exposed active catalyst material, thereby producing well aligned arrays of separated CNTs of desired length for a given application, for example, long continuous CNTs from 100% of the pores.

Optionally, post annealing of CNTs may be conducted to improve graphitization as is known in the art.

As described earlier, this growth process helps ensure that the active growth sites are at the tips of the CNTs, and are thus consistently exposed to the feedstock gas. This minimizes (if not eliminates) the tendency for the production rate to drop over time, and improves overall CNT yield as compared to the related art.

As a result, this type of CNT growth facilitates flat panel displays and other microelectronics requiring well-graphitized, aligned CNTs of equal length. This process also facilitates direct electrochemical deposition of nano particles of hydrogen getter elements such as Mg, Li, Pt, Pd etc. on the arrays of CNTs. These CNT arrays provide both the material on which the electrochemical charging of hydrogen is attained and as part of the physical structure comprising the hydrogen storage and delivery system.

Anodized Template with Inter-Connected Pores for Self-Assembled CNT Arrays

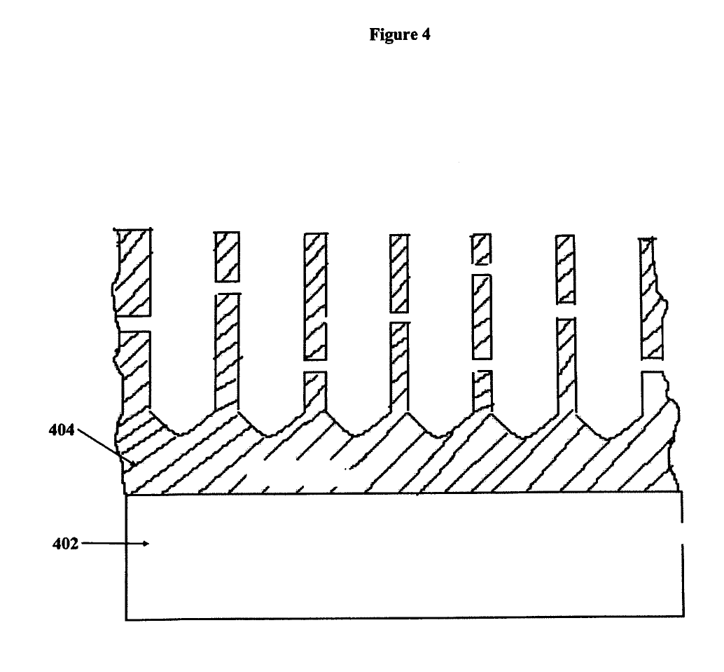

FIG. 4 illustrates a cross-sectional view of AAO template with internal pores for assembling CNTs according to another embodiment of the invention. In some applications, the AAO template may be removed. In the absence of the template structure, the CNTs would be held together by Van Der Waals forces. In order to have a self-assembled array of CNTs, the invention constructs AAO templates with internal pores as shown in FIG. 4. The procedure involved in preparation of such a template is almost similar to that described in the section “Method of Template Production” above, with the variation in the starting substrate material and intermediate dissolution process to create internal pores.

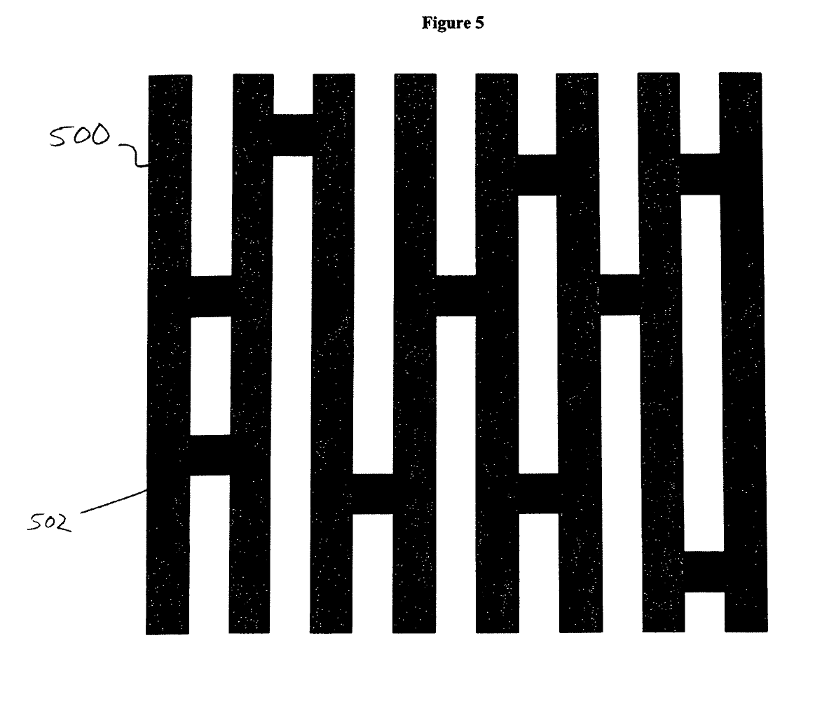

Referring to FIG. 4, a binary aluminum alloy substrate 402 will be anodized in stead of a pure Al substrate. The alloy can be a bulk material or just surface alloyed with process like ion implantation. Binary or tertiary alloyed surface can be prepared by implanting elements like Cr, Ni, Cu, Mn, Fe, Mg, Si, and the like. The alloying element will be oxidized and be present as a cation (such as Cr³⁺) in the barrier layer 404. Anodization at higher potential will further oxidize Cr³⁺ to Cr⁶⁺ and eventually it will dissolve creating lateral pores interconnecting longitudinally. Depending on the mobility of solute cations and anodization time, the location of the lateral pores can be controlled. Further incorporation of nanosize precipitates such as Mg₂Si, Ni₃Al in the aluminum oxide longitudinal pore walls and subsequent dissolution of such precipitates also will form an interconnecting nanopore structure. Referring to FIG. 5, CNT arrays grown out of these interconnecting pores are schematically represented in FIG. 5. CNTs 500 are grown and aligned via graphite blocks 502. The graphite blocks are formed in-situ via because there is no catalyst in the side-walls of template 400 of FIG. 4. The template 400 has been removed by techniques known in the art, such as, for example, chemical wet etching using sodium hydroxide.

Template for Production of Y-type CNTs

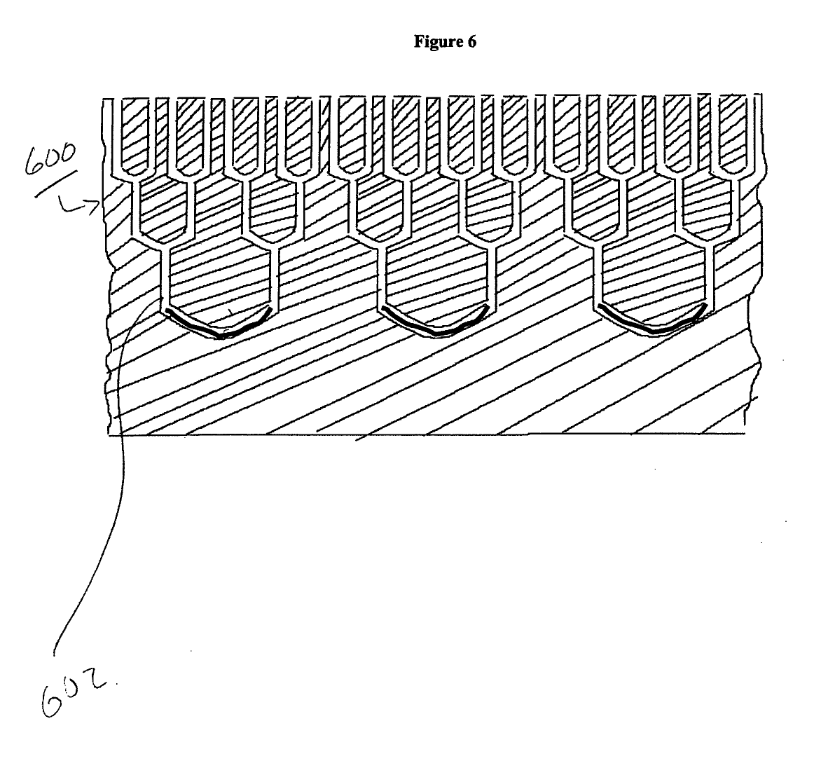

FIG. 6 illustrates a cross-sectional view of a template having pore density gradient for production of Y-type CNTs according to another embodiment of the invention. More specifically, in some micro-fluidic type applications Y-type CNTs may be required. This section outlines a brief procedure for production of templates having Y-type pore configuration for use in the growth of Y-type CNTs.

In this embodiment, pure aluminum substrates have been anodized at three different potentials to form an AAO template 600. More specifically, an increase in voltage at each step for varying amounts of time can be carried out in electrolytes such as chromate-borate, oxalic acid or sulfuric acid solutions, thereby forming Y-type pores structure 602. The pore density is inversely proportional to the voltage, that is lower the voltage higher the pore density. Initially when porous oxide is formed at lower voltage, the pore diameter is small and pore density (number of pores per unit area) is high. Stepping up the potential by, say 40% decreases the pore density and increases the diameter. As the aluminum oxide film grows at the metal/oxide interface and the growth is continuous with previously formed porous structure, Y-type pores are formed. Referring to FIG. 6, the dark lines at the bottom channels represent the deposition of catalyst for CNT growth. The catalyst deposition can be performed via pulse reversed-electrodeposition, as described herein.

CNTs were grown from the catalysts (not shown). The Y-type or branched CNTs possess different electronic properties from those of straight CNTs. This difference in electronic properties are attributed to the five and seven membered rings of the junctions in stead of regular six membered rings observed in straight tubes. Multi-junction CNTs can be building blocks for sensors, nanoelectronic devices and nanofluidic applications.

Growth of Long Aligned Carbon Nanotube Arrays

In a preferred embodiment of the invention, carbon nanotubes (CNTs) and smooth carbon nanotube structures were grown by chemical vapor deposition (CVD) via anodic aluminum oxide (AAO) templates which had cobalt electrodeposited from the AAO pore bottoms on up. The AAO pore dimensions, CVD conditions, and CNT growth results are listed as Ref. Inv. in Table 2 below:

TABLE 2

| Template | | | | | Catalyst Reduction | | | | Chemical Vapor Deposition | | | |
|---|---|---|---|---|---|---|---|---|---|---|---|---|
| | | depth to | catalyst | unfilled | | flow | | | | flow | | |
| | | catalayst | diameter | diameter | reducing | rate | time | temp | growth | rate | time | temp |
| Ref | catalyst | μm | nm | nm | gas | sccm | Hr | C | gas | sccm | min | C |
| 1 | Co | 0.05 | 55 | 75 | NA | 100 | NA | NA | CO | 100 | 720 | 700 |

| | SEM Characterization | | | | | TEM Characterization | | | | | |
|---|---|---|---|---|---|---|---|---|---|---|---|
| | | beyond | length | diameter | | | | | wall | number | interwall |
| | CNTs stop at | mouth | out | out | CNT | catalyst | Graph- | CNT | thick- | of | spacing |
| Ref | Pore mouth | % | μm | nm | tips | fate | itization | core | ness | shells | nm |
| 1 | no | 100 | 11 | 60 | closed | xx | G | H | 18-20 | 50-55 | xx |

Table 1 is a summary of published reports of CNT growth by CVD via AAO templates where CNTs grew beyond the AAO pore confines utilizing AC catalyst electrodeposition. This embodiment of the invention, is described in Table 2 as Ref. Inv. and as shown from this table smooth continuous CNT structures having at least an order of magnitude larger diameters than those obtained by the other researchers in the related art were obtained. In addition, the techniques of this invention were simplified by reducing numerous processing steps by utilizing computer controlled pulse-reverse electrodeposition.

The large diameter smooth CNT structures of the invention is attributed in part to the way the metal catalyst is electrodeposited to the bottom of the AAO pores. To obtain the large diameter smooth CNT structures and CNTs growth via AAO templates, as in the preferred embodiment, CNTs nucleated in all of the AAO pores and at the same moment, by a metal catalyst. More specifically, the metal was electrodeposited via computer controlled pulse-reverse electrodeposition, which can provide control over metal grain size, control over crystallographic orientation, prevent hydrogen evolution (which has detrimental effects on catalyst deposition rates and structural continuity), and provide an even deposit (nanometer scale) in the desired pores, preferably all pores. Also, impurities from plating solution remained in the solution and not in the plated metal. In the related art, the AC electrodeposition method used in all of the work shown in Table 1 did not have the ability to control the preferred metallic characteristics and prevent hydrogen evolution listed above and there may be an unequal filling of catalyst from pore to pore without additional non-economical and complex processing steps.

To achieve the preferred characteristics, the AAO template was prepared using conventional pulsed reversed-electrodeposition apparatus in the art. A process was performed, for example, using a computer-controlled, pulse-reverse power supply, optionally with simultaneous current and voltage control in order to obtain an even electrodeposit of metal catalyst with control over the characteristics listed above. This type of plating technique was used in, Nielsch, et. al., Uniform Nickel Deposition into Order Alumina Pores by Pulsed Electrodeposition, Adv. Mater., 2000, 12. No. 8, which is hereby incorporated by reference as if fully set forth herein, for the complete filling of AAO pores with single crystal nanowires of nickel, cobalt, or iron for research on photonic crystals and magnetic studies. The method does not appear in the literature for CNT growth.

More specifically, the computer controlled pulse-reverse current and voltage control apparatus is commercially available. For example, a preferred apparatus for the computer controlled reversed pulsed-electrodeposition was obtained from TCD Teknologi ApS of Denmark. This apparatus was utilized to allow for a cathodic waveform and pulse time adjustments to deposit catalyst starting at the AAO pore bottoms via short, relatively high current density cathodic pulses, to nucleate metals (e.g., cobalt) at most of the AAO pore bottoms. The cathodic pulse is timed to stop before the metal ions in solution at the existing plated metal/metal solution interface are exhausted, preventing hydrogen formation, which would have an ill effect on the catalyst deposit. An anodic waveform and pulse time were used to discharge the capacitance of the barrier layer, immediately stop cobalt electrodeposition, and to selectively de-plate any impurities from the just plated layer of metal to leave only metal particles of a desired grain size range, and keep the metal face clean for the next cathodic pulse to lay down the next layer with the same crystallographic orientation. Finally, a rest time of up to about one second is allowed before the next cathodic pulse to replenish metal ions at the plated metal/metal solution interface.

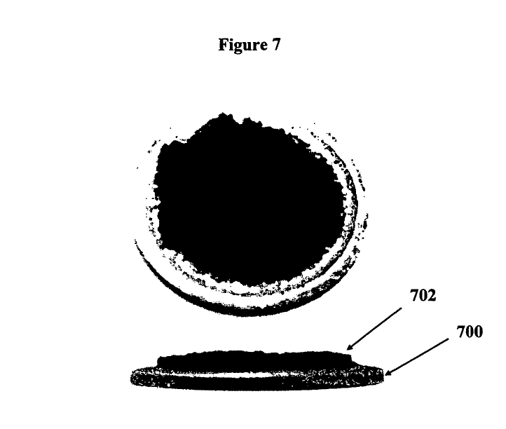

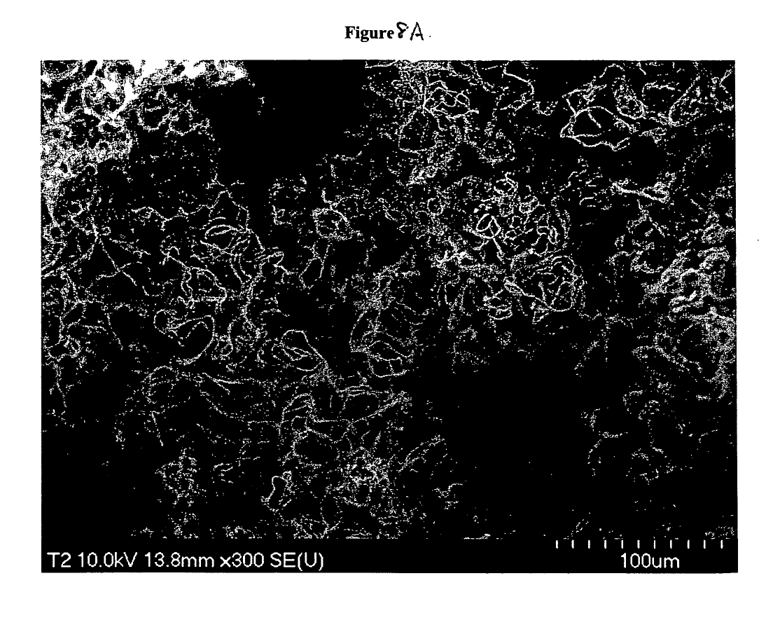

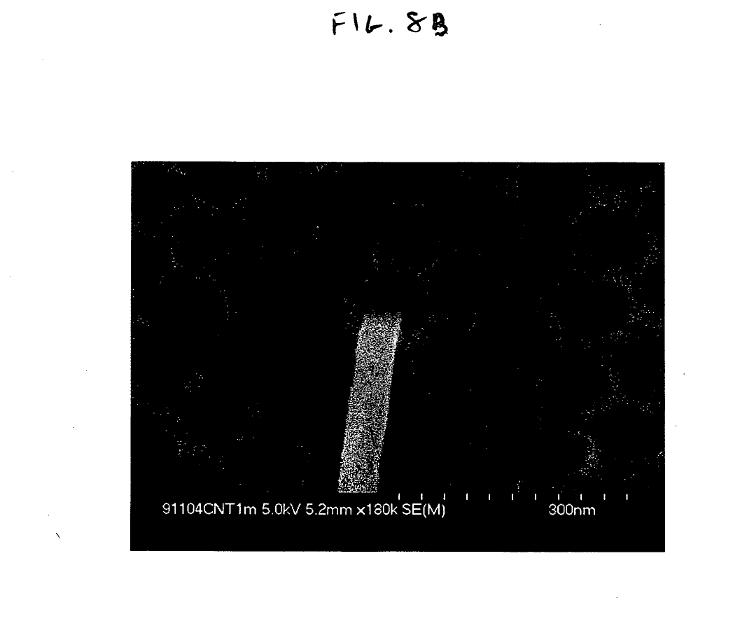

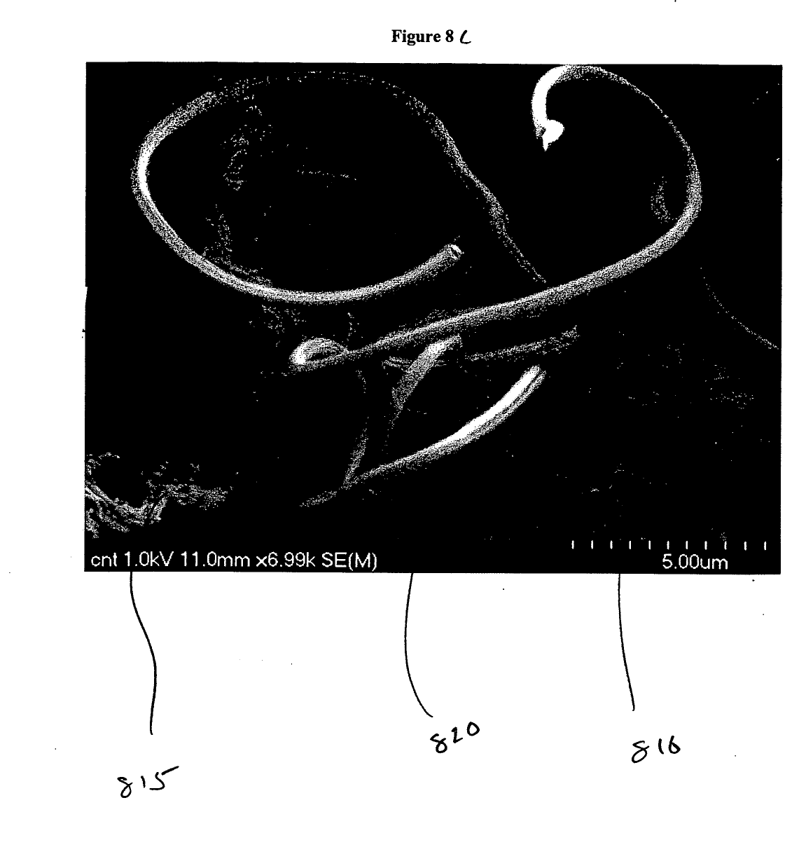

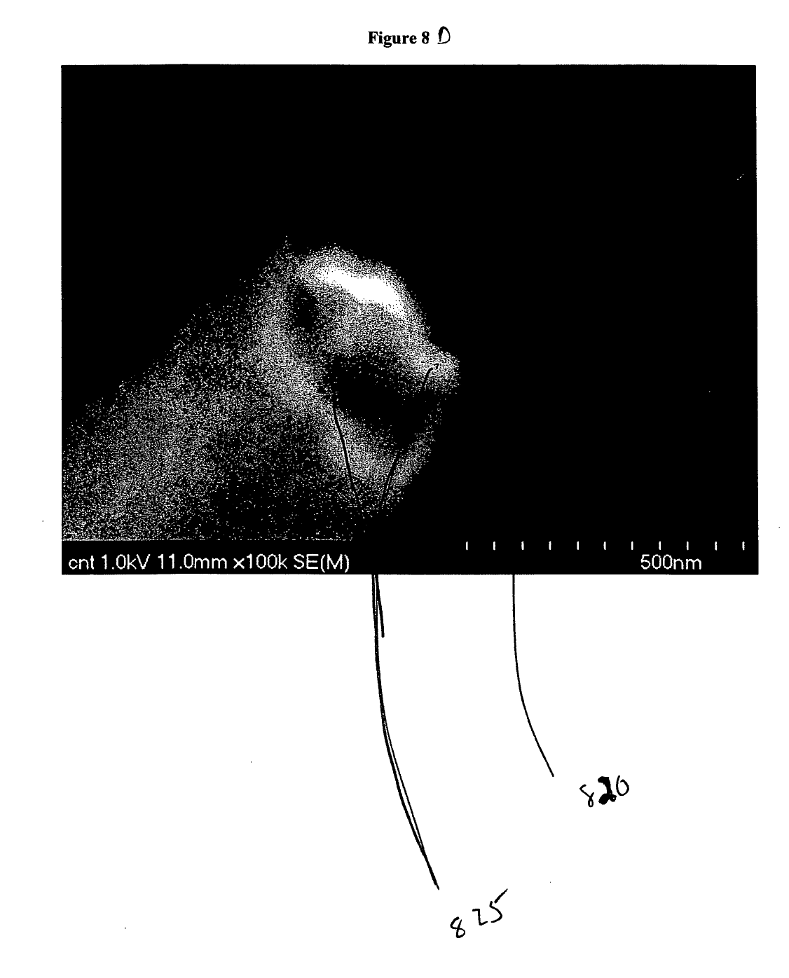

As an illustrative example, one set of electrodepostion conditions used for the invention were about 300 g/l cobalt sulphate heptahydrate, about 45 μl boric acid, at about pH 4.5, at about 35° C., cathodic pulse of about 8 milliseconds, anodic pulse of about 2 milliseconds, and rest time of about 0.6 seconds. Total electrodeposition time of about 10 minutes filled the AAO pores to a length of about 500 nm. FIGS. 7 to 8(d) show that these electroplating conditions as used in the invention produced a metallic and very active catalyst that did not require the one hour reduction under hydrogen at 500° C. as the related art required in Table 1.

More specifically, FIG. 7 shows two macroscopic images of the CNTs grown as per Ref. Inv. in Table 2. The AAO pores 702 were about 1 micron in length and about 50 nm in diameter. The CNTs were initiated by the cobalt catalyst and quickly grew far beyond the AAO template. The thickness of the aluminum substrate 700 plus the AAO pores 702 is about 1 mm. It can be seen that the height of the CNT mass is also about 1 mm. The growth conditions are detailed in Table 2 under Ref. Inv. The 10 minute growth time produced about 10 mg of growth, from an AAO template surface area of about 1 cm².

Referring to FIG. 8(a) at 300× magnification clearly reveals strands of grown CNT material. Referring to FIG. 8(b), it is shown that CNTs growing out of AAO pores in a small region where only a few of the AAO pores were active and the CNT growth did not obscure the pores from viewing.

Referring to FIG. 8(c), which is unlike any other seen in the related art there are three CNT structures to consider. The lower right region 810 shows mostly CNTs of about 25-50 nm in diameter. These have grown out of the AAO pores and did not join with other CNTs as they grew in length. The lower left 815 region shows a structure which has been described in literature as CNT ropes. The middle region of the figure shows many of the 25-50 nm CNTs growing at about the same rate and held together by Van der Waal forces to give an unusually smooth CNT structure 820 having a “rope” type shape. The diameter of the CNT structure 820 was about 500 nm, which has not been seen in literature.

More specifically, the CNT structure 820 seen here is the thick and smooth cylindrical material snaking its way through most of the image. If one looks closely, the individual 25-50 nm CNTs of region 810 can be seen. These smaller CNTs grew along side one another, at the same rate, in contact along their lengths, to create a smooth, dense, monolithic mass of CNTs or CNT structure 820.

Additionally, thick CNT ropes up to 20 cm long have been reported in the literature of the related art. However, in the related art the ropes are not dense and monolithic as shown in region 815. Rather, in the related art, they have many areas along their lengths that are not touching one another, thereby providing a structurally weaker material. The CNT growth system of the invention achieves CNTs growth from the AAO template and at a similar rate, remaining straight, attracted along their equal lengths, as they grown, by Van der Walls attraction, as an even, dense, monolithic mass that can be grown in large sheets and cylinders for use as a superior strength and light weight material. Ultimately, complex parts may be fabricated by growing CNTs from templates with desired designs. These will be superior to machined parts from inferior materials. Referring to FIG. 8(d), illustrating the CNTs structures 820 at a magnification at 100,000× some of the 25-50 nm CNTs making up the CNT structure 820 are seen extending from the fat CNT structure 825.

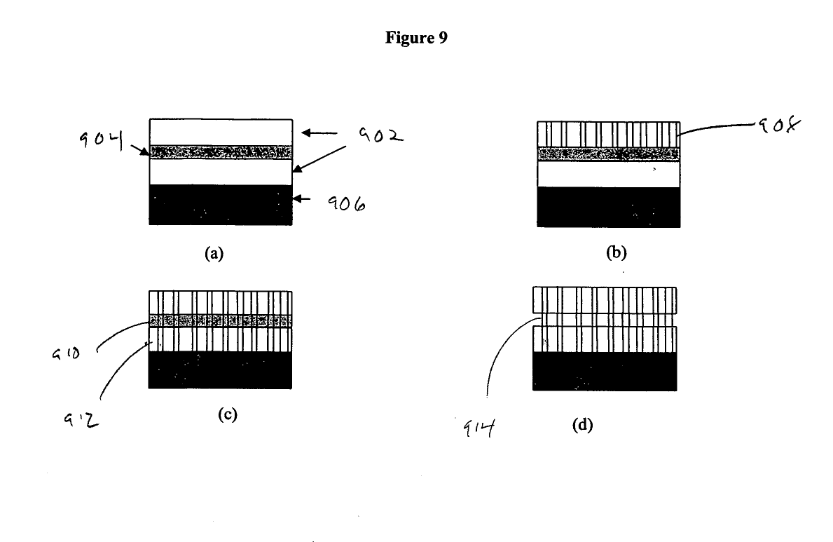

FIGS. 9(a)-9(d) illustrate a multilayered template according to another embodiment of the invention. It is known that the electrical properties of CNTs depend on their chirality. Twisting of CNTs at will allows these properties to move from semimetallic to metallic, to semiconducting. Referring to FIG. 9(a), illustrating alternate layers of aluminum 902 are formed by known techniques (e.g., sputtered) on silicon substrate 906. The aluminum layers 902 are separated by a with middle metal layer 904 that can be easily dissolved in an acidic solution (e.g., cu material). The thickness of the middle metal layer 904 may be 10 nm. In FIG. 9(b), the top aluminum layer is anodized 908 through its thickness. In FIG. 9(c), dissolution of exposed Cu surface 910 is performed followed by subsequent anodization of bottom Al layer 912. In FIG. 9(d), dissolution of middle Cu layer 914 is performed, thereby enabling rotation of top and bottom templates. More specifically, the template is put in a solution (e.g., nitric acid) to dissolve the middle layer 910, leaving a void (e.g., 10 nm) between two anodized layer with their pores still lined up as the perimeter of the template is solid. Finally, a catalyst deposition is performed and CNTs are grown as described herein throughout (not shown). For example, the CNTs may be grown by a CVD process using carbon monoxide at 100 sccm at 650° C. for about 1-5 minutes.

The CNTs bridge the gap between anodized layers to give a material where the two halves can be twisted as needed to change the electrical properties (electronic switching/computing as well) of the CNTs. This layered technique is also useful for extrusion growth of CNTs.

Doping of Directionally Grown CNTs for Electronic and Hydrogen Storage Applications

CNTs can act as a metallic conductor or semiconductor depending on their diameter, chirality, and temperature. Doping of elements to either p or n type acting as an electron acceptor or donor would change the semiconductivity of CNTs. Their nanosized diameter and microns (or more) length make them dimensional conductors. This one-dimensional conducting property along with modification of semiconductivity by doping different elements could be tailored to make various electronic circuits for potential applications such as gas sensors, fuel cells, and the like. CNTs have been considered as a potential hydrogen storage material. Hydrogen is stored at the surface or in the cavity of the CNTs as a condensed monolayer. As the surface to mass ratio is higher for single-walled CNTs versus multi-walled CNTs, more hydrogen could be stored by controlling the morphology and size of the CNTs. By doping hydrogen absorbing materials in the CNTs, the hydrogen storage could be maximized as large surface area is available for both adsorption and absorption. In order to have better mass ratio of hydrogen storage it is better to use light weight hydride metals, such as, for example, Mg and Li. Various metals like Pt, K, Li, Au, and the like, have been doped in CNTs for catalytic applications. Controlled arrays of CNTs and doped with suitable hydride forming elements for hydrogen storage applications have the following advantages:
Controlled array of CNTs may potentially give better access for diffusion or other reaction paths so that better reactivation kinetics may be observed.
Different catalyst elements can be doped in CNTs, i.e., for hydride forming as well as for hydrogen desorption.
Possibilities of large surface area, defined reaction sites, and presence of the required catalyst by doping, potentially reduces the operating temperature for hydrogen desorption.
Normally, hydrogen sorption/desorption reaction on magnesium hydride occurs in the temperature range of 350-400° C. Deposition of nanosized Mg particles on CNTs could decrease this reaction temperature to 150° C.
The reported maximum storage capacity for magnesium hydride is about 7.3 wt % and for CNTs it varies widely from about 2-10%. Higher hydrogen storage capacity in CNTs has always been questioned, because of experimental errors. In addition, reported higher storage capacity pertains to sorption at liquid nitrogen temperature and desorption at higher temperatures than commercially relevant conditions. The proposed composite of Nanosized Mg in CNT arrays may have hydrogen storage capacity of about 5-10 wt % at lower operating temperatures comparable to that of commercially relevant conditions.
In composite of Mg-CNT arrays hydrogen storage would occur by two mechanisms viz., i) by physisorption on CNTs, and ii) by chemisorption on Mg.

The CNTs grown using the procedure of the invention as described in the section above under “Procedure for Controlled and Directional Growth of Long-range Carbon Nanotubes.” The invention enables electrochemical deposition of hydrogen getter elements as well as electrochemical charging/discharging of hydrogen. Hydrogen getter elements like Mg may be deposited via physical vapor deposition using techniques known in the art.

For electronic and gas sensor applications, various elements such as K, Li, Br, Pt, Au, O, N, B, and the like, will be doped using physical or chemical vapor deposition methods on CNTs grown out of the templates described in sections above.

Extrusion Like Fabrication Method for Producing Meters Long CNTs

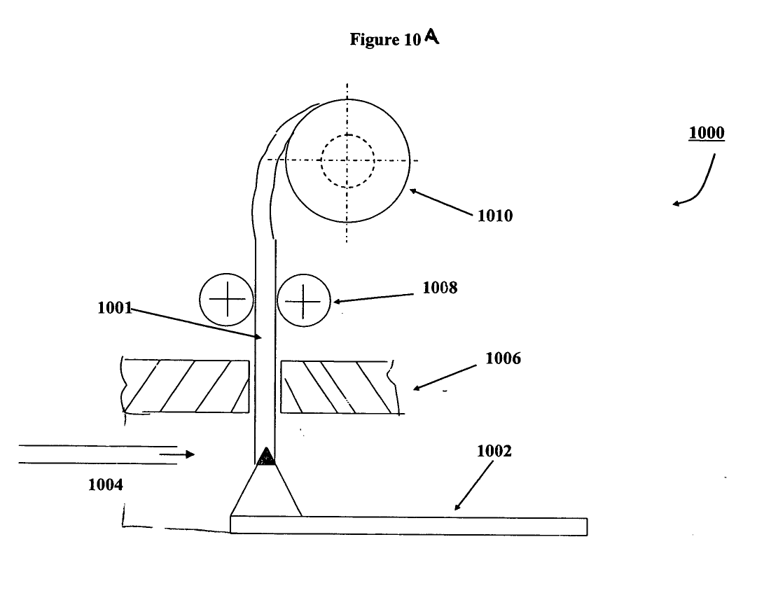

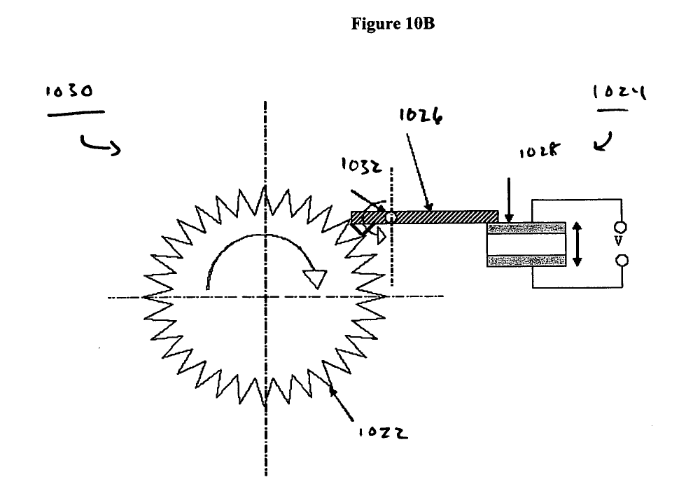

FIG. 10(a) illustrates an apparatus generally depicted as reference 1000 and method to produce continuous carbon nanotubes according to another embodiment of the invention. More specifically, the apparatus generally depicted as reference 1000 allows for CNTs to be grown without tangling, that is, they may be wound up around a spool for future use. In this embodiment, an commercially available AFM probe-cantilever tip 1002, plated with a nanocatalyst particle to grow a carbon nanotube may be used. The AFM probe-cantilever type is known in the art.

A gas supply 1004 is supplied into the apparatus 1000 for supplying gas for CVD growth of CNTs. A nanoporous template 1006 prepared according to the invention serves as a guide for the nanotubes 1001. For example, the nanoporous template of FIG. 3(c), prior to deposition of catalyst. More particularly, the substrate (e.g., aluminum) has been completely dissolved after anodization, for example, in a mercuric chloride solution thereby leaving only the uniform porous membrane to serve as a guide for subsequently grown CNTs 1001. Conveyance rollers 1008 for pushing CNT is arranged above the nanoporous template 1006. A spool 1010 for receiving and/or pulling continuous CNT grown in the apparatus 1000 and may be arranged above the spool 1010. The movement of rollers 1008 and spool 1010 are synchronized with the growth rate of CNTs to provide spooling of the CNTs. The entire assembly is encapsulated in a chamber to maintain a CNT growth temperature of about 650-700° C.

Referring to FIG. 10(b), a mechanism for winding spool generally depicted as reference number 1030 is arranged outside the chamber. The spool 1010 and the ratchet 1022 to drive the spool 1010 are integral parts. More specifically, in the method a single carbon nanotube is grown on a catalyst deposited tip of an AFM probe 1002 using a CVD method. The growing CNT 1001 may be guided through a nanoporous template 1006 and optional conveyance rollers 1008 or may be gravity fed to the spool 1010. In this embodiment, the rollers 1008 push the CNT 1001 at a rate equivalent to the CNT growth rate. The conveyance rollers 1008 also can be actuated by the mechanism generally depicted as reference number 1030, shown in the figure, if necessary. The pushed CNT 1001 is spooled by another roller which is rotating at a linear velocity equivalent to that of the CNT growth rate. A thin film piezoelectric actuator-ratchet assembly 1024 enables the rotation of the pulley 1010. Fabrication of such thin film piezoelectric actuators that can give linear displacements in nanometer resolution is known in art, Wong, et al., Analog tunable gratings driven by thin-film piezoelectric microelectromechanical actuators, Applied Optics, 42 (2003) 621-626, which is hereby incorporated by reference as if fully set forth herein. More specifically, by controlling the voltage and the thickness of the lead-zirconate-titanate (PZT) oxide film, nanometer lever displacements can be easily obtained. That is, application of square wave voltage pulse expands or contracts the PZT film, depending on the voltage polarity. When the PZT 1028 expands, it actuates the cantilever 1026. The linear expansion of the PZT 1028 is transformed to oscillatory motion of the cantilever 1026. The tip of the cantilever in turn rotates the ratchet by pushing one tooth down completely or partially (depending on the magnitude of the pulse) for each potential pulse applied to the PZT. The rotation is shown in by the arrow and about a pivot point 1032. Adjusting the voltage amplitude can minimize contraction of the PZT and this contraction (if any) does not rotate the ratchet in the opposite direction because of the shape of the tooth. The voltage pulse frequency determines the velocity of the ratchet. The growth rate and movement of the roller is synchronized precisely at Angstrom level.

Using this method meters of long continuous CNTs can be produced. The continuous CNTs produced using the above method can be used for space elevator application as load bearing belts. The above CNTs can be used for manufacturing fabrics having ultra high tensile strength which can be used for ballistic protection and as blast containers.

The continuous CNTs produced according to the invention may be used for space elevator application as load bearing belts. The above CNTs may also be used for manufacturing fabrics having ultra high tensile strength which can be used for ballistic protection and as blast containers has been proposed.

It will be apparent to those skilled in the art that various modifications and variation can be made in the present invention without departing from the spirit or scope of the invention. Thus, it is intended that the present invention cover the modifications and variations of this invention provided they come within the scope of the appended claims and their equivalents.

1. An anodized aluminum oxide template for carbon nanotube growth, comprising:
anodized aluminum substrate;
a plurality of pores arranged in the anodized aluminum substrate;
a plurality of catalyst particles arranged substantially uniformly in the plurality of pores to provide nucleation sites for carbon nanotube growth;
a plurality of carbon nanotubes arranged within the pores; and
a metal material arranged over the anodized aluminum substrate and covering a plurality of carbon nanotubes.

2. The anodized aluminum oxide template of claim 1, wherein the average pore diameter is about 50 nm.

3. The anodized aluminum oxide template of claim 1, wherein the anodized aluminum substrate comprises an aluminum alloy.

4. The anodized aluminum oxide template of claim 1, wherein the plurality of pores have a density of about 10¹⁰/cm².

5. The anodized aluminum oxide template of claim 1, wherein an average interpore center-to-center distance between at least two of the plurality of pores is about 100 nm.

6. The anodized aluminum oxide template of claim 1, wherein the plurality of catalyst particles comprise cobalt deposited with pulse-reverse electrodeposition.

7. The anodized aluminum oxide template of claim 1, wherein the plurality of carbon nanotubes comprises a first portion having a first composition and a second portion having a second composition, wherein the first composition is different than a second composition.

8. The anodized aluminum oxide template of claim 7, wherein the second composition comprises graphitized carbon nanotubes.

9. The anodized aluminum oxide template of claim 8, wherein the first composition comprises carbon nanotubes having a graphitization less than the second composition.

10. The anodized aluminum oxide template of claim 1, wherein the plurality of catalyst particles are selected from the group metals consisting of Co, Ni, Fe, transitional metals, and combinations thereof.

11. The anodized aluminum oxide template of claim 1, wherein the metal material comprises transition metals selected from the group consisting of nickel, platinum, chromium and combinations thereof.

12. The anodized aluminum oxide template of claim 1, further comprising a protective layer arranged on the barrier layer.

13. A method of fabricating anodized aluminum oxide template, comprising the steps of:
annealing an aluminum material;
electropolishing the annealed aluminum material;
performing a first anodizing of the aluminum material and removing the first anodized material;
performing a second anodizing of at least a portion of the aluminum material to form an anodized aluminum layer and to form a plurality of pores in the anodized aluminum layer;
electrodepositing a plurality of catalyst particles arranged substantially uniformly in the plurality of pores to provide nucleation sites for carbon nanotube growth;
growing carbon nanotubes within the plurality of pores; and
forming a metal layer on the anodized aluminum layer covering the carbon nanotubes.

14. The method of claim 13, wherein the step of growing the carbon nanotubes step within the plurality of pores, comprises:
applying a first gas for a first time period to grow a first portion of the first set of carbon nanotubes having a first composition; and
applying a second gas for a second time period to grow a second portion of the first set of carbon nanotubes having a second composition.

15. The method of claim 14, wherein the first time period is longer than the second time period.

16. The method of claim 13, wherein the annealing aluminum step comprises annealing aluminum material under atmosphere selected from the group consisting of helium and argon at a temperature of about 500° C. for about 3 hours; the electropolishing step comprises contacting the annealed aluminum material with an acid bath solution of pechoric acid/ethanol at a ratio of about 1:3; and the second anodizing step comprises anodizing the electropolished annealed aluminum material in about a 0.3 M oxalic acid solution at a voltage of about 40 DC to form a nanoporous anodized aluminum oxide Al₂O₃ layer.

17. The method of claim 13, wherein the electrodeposition step is a computer controlled pulse-reverse electrodeposition with simultaneous current and voltage control.

18. The method of claim 13, further comprising the step of:
controlling an average diameter of the plurality of pores to about 50 nm.

19. The method of claim 18, wherein the controlling an average diameter step comprises controlled widening the entire pore of the plurality of pores in about a 0.3 M oxalic acid solution.

20. A method of forming carbon nanotubes, comprising the steps of:
providing an aluminum material having a first surface and a second surface opposite said first surface;
anodizing the first surface of the aluminum material to form a plurality of pores;
depositing a catalytic material into the plurality of pores to provide nucleation site for a first set of carbon nanotubes and a second set of carbon nanotubes at bottom of the plurality of pores;
growing the first set of carbon nanotubes from the catalytic metal to reach about the top portion of the plurality of pores;
forming a metal layer over the anodized aluminum material and covering the first set of carbon nanotubes;
forming a protective barrier layer over the metal layer;
removing a portion of the aluminum material on the second surface to expose at least a bottom portion the catalyst metal; and
growing a second set of carbon nanotubes at the exposed portion of the catalytic metal.

21. The method of claim 20, further comprising the step of:
controlling the size of the plurality of pores to have an average diameter of about 50 nm.

22. The method of claim 20, wherein the second set of carbon nanotubes are grown in substantially uniform bundles.

23. The method of claim 20, wherein the growing the first set of carbon nanotubes step comprises:
applying a first chemical vapor deposition with a first gas for a first time period to grow a first portion of the first set of carbon nanotubes having a first composition; and
applying a second chemical vapor deposition with a second gas for a second time period to grow a second portion of the first set of carbon nanotubes having a second composition, wherein the first composition is different than the second composition.

24. The method of claim 23, wherein the first gas comprises carbon monoxide and the second gas comprises acetylene and argon.

25. The method of claim 20, wherein the growing the second set of carbon nanotubes step comprises:
applying chemical vapor deposition with a carbon monoxide gas to grow the second set of carbon nanotubes.

26. The method of claim 20, wherein the second set of carbon nanotubes comprises a long continuous carbon nanotube being substantially well-graphitized.

27. The method of claim 20, wherein the forming a metal layer step comprising sputtering a transitional metal material.

28. The method of claim 27, wherein the metal material comprises a transition metal selected from the group consisting of nickel, platinum, chromium and combinations thereof.

29. The method of claim 20, wherein the forming catalytic material step comprises performing computer controlled pulse-reverse electrodeposition of a catalytic metal with simultaneous current and voltage control.

30. The method of claim 20, wherein the catalytic metal is selected from the group metals consisting of Co, Ni, Fe, transitional metals, and combinations thereof.

31. The method of claim 20, wherein the aluminum material comprises an alloy mixture.

32. The method of claim 20, further comprising:
doping the aluminum material by implanting elements selected from the group consisting of Cr, Ni, Cu, Mn, Fe, Mg, Si, and combinations thereof.

33. The method of claim 20, further comprising:
interconnecting at least two of the plurality of pores in the anodizing step.

34. The method of claim 20, wherein at least a portion of the plurality of pores are formed in a Y-type shape.

35. A method of fabricating anodized aluminum oxide template, comprising the steps of:
providing a substrate;
forming an aluminum material on the substrate;
anodizing at least a portion of the aluminum material to form an anodized aluminum layer and to form a plurality of pores in the anodized aluminum layer; and
pulse reverse-electrodepositing a plurality of catalyst particles arranged in the plurality of pores to provide nucleation sites for carbon nanotube growth.

36. The method of claim 35, further comprising:
annealing the aluminum material and polishing the annealed the annealed aluminum material prior to the anodizing step.

37. The method of claim 35, further comprising:
growing carbon nanotubes from the plurality of catalyst particles; and
forming a metal layer on the anodized aluminum layer covering the carbon nanotubes.

38. The method of claim 35, wherein the substrate is selected from the group consisting of plastic, semiconductor, metal, and combinations thereof.

39. The method of claim 35, further comprising:
forming a metal material on the aluminum material prior to the anodization step; and
forming a second aluminum material on the metal material prior to anodization step.

40. The method of claim 35, wherein the metal material comprises copper.

41. A carbon nanotube growth kit, comprising,
a first and second set of carbon nanotubes formed by the process of claim 20.
